(19) United States
(12) Patent Application Publication
(10) Pub. No.: US 2026/0344827 A1
(43) Pub. Date: Nov. 5, 2026

(54) AUTO-CALIBRATED SMART INSOLE FOR LONG-TERM MEASUREMENT OF GROUND REACTION FORCE

(71) Applicant: University of New Hampshire, Durham, NH (US)

(72) Inventors: Diliang CHEN, Newmarket, NH (US); Femi John OLUGBON, Durham, NH (US)

(21) Appl. No.: 19/487,763

(22) PCT Filed: May 24, 2024
(86) PCT No.: PCT/US2024/031022

§ 371 Date: Nov. 25, 2025

Related U.S. Application Data
(60) Provisional application No. 63/504,521, filed on May 26, 2023.

(51) Int. Cl. A43B 3/44; A43B 17/00; A61B 5/00; A61B 5/103; A61B 5/11

(52) U.S. Cl. CPC A43B 3/44; A43B 17/00; A61B 5/1038; A61B 5/112; A61B 5/6807; A61B 5/7267; A61B 2560/0223; A61B 2562/0247; A61B 2562/046

(57) ABSTRACT

Wearable force sensing insoles with automatic calibration. In one example, an insole apparatus includes an insole, a flexible pressure sensor array coupled to the insole and including a plurality of pressure sensors distributed over a surface area of the insole, a force plate integrated into a heel region of the insole, and an inertial measurement unit coupled to the insole.

### CROSS-REFERENCE TO RELATED APPLICATIONS

This application claims priority to co-pending U.S. Provisional Application No. 63/524,501 filed on May 26, 2023, which is hereby incorporated herein by reference in its entirety for all purposes.

### BACKGROUND

Measurement of ground reaction force (GRF) is useful in a wide variety of applications, such as healthcare, occupational safety, and sports performance. Some systems that incorporate insole-based pressure sensors suffer from several drawbacks, including high cost, bulky apparatus that is not comfortable to wear during daily life, and low accuracy in long-term measurement performance. In addition, the requirement for manual calibration before each usage largely limits widespread application of such systems. Accordingly, a number of challenges remain with respect to developing accurate GRF measurement systems.

### SUMMARY

Aspects and embodiments are directed to techniques for auto-calibrating a flexible pressure sensor array that can be incorporated into an insole to provide a force sensing mechanism for lifted load weight measurement and other applications.

As described further below, in one example, an insole apparatus includes an insole, a flexible pressure sensor array coupled to the insole and including a plurality of pressure sensors distributed over a surface area of the insole, a force plate integrated into a heel region of the insole; and an inertial measurement unit (IMU) coupled to the insole. A calibration module can be integrated into or coupled to the insole and configured to perform automatic calibration of the flexible pressure sensor array using motion measurements from the IMU and reference force measurements from the force plate. In some examples, the calibration module uses machine learning approaches (e.g., an artificial neural network) to automatically perform the calibration, as described further below.

These and other aspects are described in more detail below.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and are incorporated in and constitute a part of this disclosure. However, the figures are not intended as a definition of the limits of any particular example. In the figures, the same or similar components are represented by a like reference numeral. For purposes of clarity, every component may not be labeled in every figure. In the figures:

FIG. 1 is a flow diagram illustrating an example of stages of conditions that can occur in persons with diabetes;

FIG. 2 is a graph showing an example of ground reaction force over a person's walking stride;

FIG. 3 is an exploded view of one example of an insole apparatus according to aspects of the present disclosure;

FIG. 4 is a diagram illustrating an exploded view of an example of a wearable force plate according to aspects of the present disclosure;

FIG. 5 is a block diagram of an example of a force measurement system according to aspects of the present disclosure;

FIG. 6A is a flow diagram of an example of a calibration process for an insole apparatus according to aspects of the present disclosure;

FIG. 6B is a flow diagram of another example of a calibration process for an insole apparatus according to aspects of the present disclosure;

FIG. 7 is a graph showing various ground reaction force curves according to aspects of the present disclosure; and

FIG. 8 is a graph showing pressure sensor force estimates according to aspects of the present disclosure.

### DETAILED DESCRIPTION

Low-back pain is a prevalent health problem in workplaces. Lifting a heavy load, such as during manual material handling, is a significant cause of low-back pain. Lifted load weight, and/or load vertical location, are parameters that can be monitored and used to evaluate risk of low-back pain. However, existing systems lack reliable, convenient, accurate methods by which to monitor these parameters.

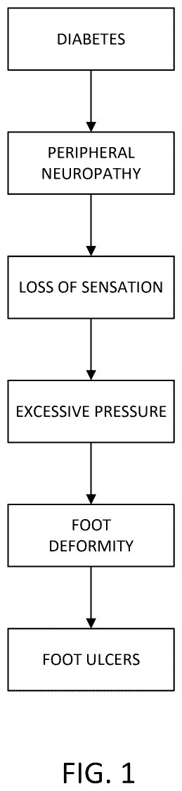

Diabetic foot ulcers are sores or wounds that most commonly occur on the bottom of the foot. Diabetic foot ulcers affect a significant number of diabetic patients and are a leading cause of amputations in diabetic patients. For example, about 85% of amputations are preceded by a diabetic foot ulcer. The 5-year survival rate for diabetes related amputation is only 40-48%. Therefore, there is a great need for, and a great benefit associated with, the prevention of diabetic foot ulcers, including detection of conditions preceding a diabetic foot ulcer. High resolution pressure measurements can be used in the detection and prevention of diabetic foot ulcers. For example, FIG. 1 illustrates that excessive pressure placed on the foot due to diabetes symptoms can cause foot deformity, potentially leading to diabetic foot ulcers. Thus, plantar pressure mapping may be used to detect excessive foot pressure, allowing for action to be taken to potentially prevent a diabetic foot ulcer from occurring. However, existing pressure mapping technologies suffer from several drawbacks that limit their use and application.

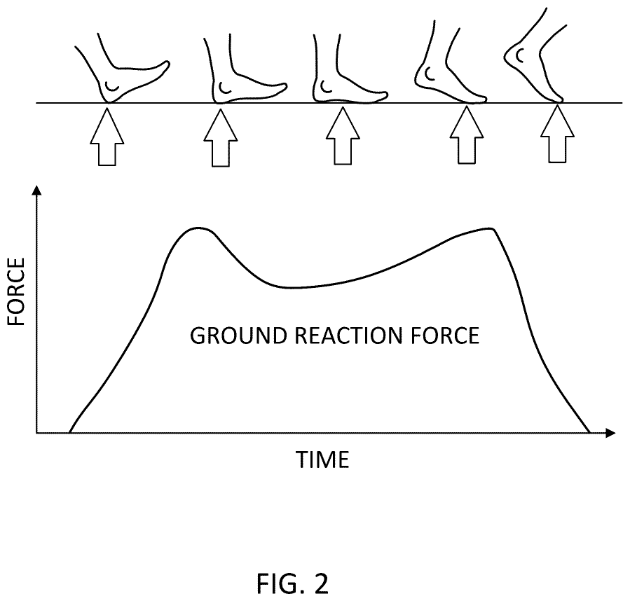

Ground reaction force (GRF) is the force exerted by the ground on a body in contact with the ground. GRF is the force that propels the human body, enabling various activities, and it reflects information about locomotion, gait, and injury risk associated with human movement. For example, FIG. 2 is a graph illustrating GRF at different stages of a person's walking gait. As a person walks, different areas of the foot are in contact with the ground over time, as illustrated in FIG. 2. Accordingly, a wearable pressure sensor, such as a pressure sensor that can be incorporated into an insole, for example, can provide GRF measurements. As shown in Table 1 below, GRF measurements can be useful in a wide variety of applications, such as healthcare (including prevention of diabetic foot ulcers), occupational safety, and sports performance.

TABLE 1
Applications of GRF measurements with varying accuracy
Accuracy and Spatial Resolution

| Low | Medium | High |
|---|---|---|
| Existence of force | Accurate total GRF | Accurate force |
| | | on individual |
| | | sensors in an array |
| Basic gait parameters, | Total plantar pressure, | High resolution pressure |
| such as stride time | weight acceptance ratio | map, center of pressure |
| Rehabilitation | Physical workload | Diabetic foot ulcer |
| assessment | Injury risk | risk detection |
| Sports performance | assessment | Balance assessment |
| optimization | Gait efficiency | Footwear and |
| Fall risk assessment | Footwear and | orthotics design |
| | orthotics evaluation | |

Accordingly, aspects and examples are directed to an insole apparatus that can be used to obtain force/pressure measurements, including GRF measurements, that in turn can be used to for a variety of applications. Some force sensing insoles use a pressure sensor array that tends to experience drift over time, leading to inaccuracies in long-term measurement performance and a need for frequent calibration. A requirement for manual calibration before each usage can present a significant barrier to the use of insole pressure sensors, and limits widespread application of the sensors. Furthermore, certain calibration methods have significant drawbacks. For example, calibration methods that are performed with specific equipment can be expensive and time-consuming. Accordingly, non-trivial issues remain with respect to using insole pressure sensors.

To address these challenges, techniques are described herein for providing automatic calibration of a flexible pressure sensor array used in a force sensing insole. According to certain examples, there are provided an insole apparatus and calibration processes to achieve automatic and accurate calibration for each sensor under the foot. Examples include an insole with built-in automatic calibrating pressure measurement capability that provides long-term measurement performance with high accuracy and an unobtrusive design that is comfortable and lower cost than some existing devices. As described further below, certain examples provide an insole apparatus that incorporates a wearable force plate to supply an absolute force measurement as reference to allow for automatic calibration of a flexible pressure sensor array that is also incorporated into the insole. The calibrated flexible pressure sensor array may then be used to measure the absolute force during various activities, which can be used to monitor lifted load weight, for example, or for other applications.

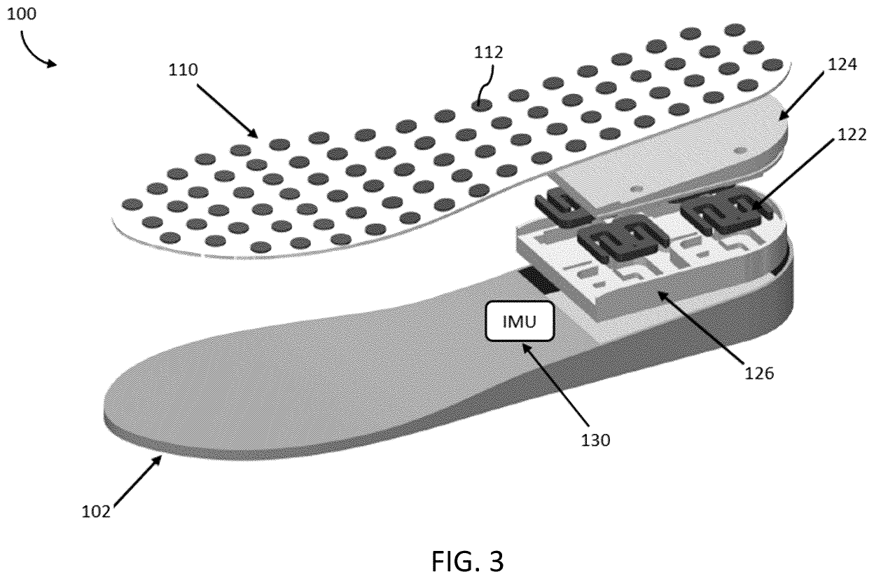

Referring to FIG. 3, there is illustrated an insole apparatus 100 according to certain examples. The insole apparatus 100 can be incorporated into an insole 102 that can be inserted into a shoe or boot. In some examples, the insole apparatus 100 includes a flexible pressure sensor array 110 that includes a plurality of pressure sensors 112. In some examples, the flexible pressure sensor array 110 may conform generally to the shape of the insole 102, such that the pressure sensors 112 are distributed over the area of the foot when the insole is worn. In other examples, the pressure sensors 112 may be distributed over only a portion of the foot area. In one example, the flexible pressure sensor array 110 includes 96 pressure sensors 112 distributed over the foot area; however, any number of sensors may be used. The flexible pressure sensor array 110 may have a thickness in a range of about 1 millimeter (mm) to 2 mm. In some examples, the flexible pressure sensor array 110 can be trimmable to fit different sizes of foot (or different sizes of the insole 102).

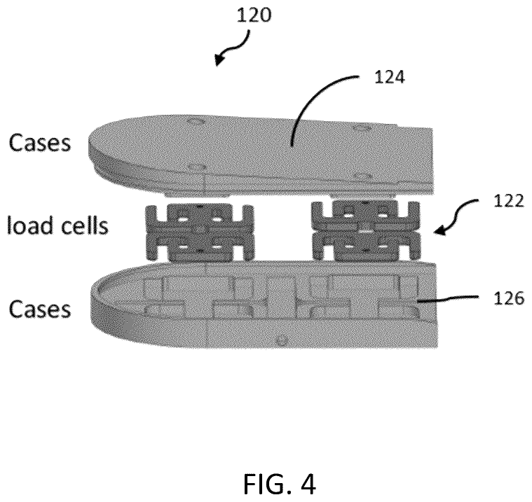

In some examples, the insole apparatus 100 further includes a wearable force plate 120 (see FIG. 4) that is positioned in the heel area of the insole 102. The wearable force plate 120 supplies an absolute force reference for auto-calibration of the flexible pressure sensor array 110, as described in more detail below.

Referring to FIGS. 3 and 4, in some examples, the wearable force plate 120 is constructed with load cells 122, which are physical transducers that can translate force into an electrical signal. In the illustrated example, the wearable force plate 120 includes a plurality of load cells 122 housed in a housing comprises upper and lower casings 124, 126, respectively, that can be integrated into the heel area of the insole 102. The wearable force plate 120 may be constructed using accurate and stable micro load cells 122. In one example, four micro load cells 122 are placed on four corners of the wearable force plate 120 to ensure that load forces can be stably applied to each load cell during different activities. Micro load cells are frequently used for building body weight scales, which have good accuracy (±0.1%), repeatability (±0.1%), and measurement range (up to 75 kg/load cell). Accordingly, the micro load cells can be used for calibrating the flexible pressure sensor array 410. In addition, micro load cells are thin (2.5 mm) and lightweight (9.7 g), which makes them suitable for building wearable force plates.

In some examples the wearable force plate 120 is integrated into the insole 102 for long-term measurement, and accordingly, the design and construction of the force plate 120 may consider both measurement accuracy and wear comfort. To provide good measurement accuracy, the wearable force plate 120 is configured and positioned in the insole 102 such that all the load forces go through the part of the load cell where its deformation is sensed. The wearable force plate 120 may be rigid. In particular, the upper and lower casings 124, 126 that protect the load cells 122 may be made of a rigid material. To provide wear comfort, examples of the wearable force plate 120 are thin, lightweight, and positioned in the foot area that does not bend. Accordingly, in some examples, the wearable force plate 120 is integrated into the heel area of the insole 102, as shown in FIG. 3, because the heel area of the human foot generally does not bend during different activities, including walking.

Experimental results have shown that an example of the wearable force plate 120 in a normal sneaker has a good accuracy, with a Mean Absolute Error (MAE) of 0.202 Newtons (N), in measuring both static and dynamic forces when compared with the gold standard force plate.

Still referring to FIG. 3, in some examples, the insole apparatus 100 further includes an inertial measurement unit (IMU) 130. In one example, the IMU is a 6-axis IMU that integrates a 3-axis accelerometer with a 3-axis gyroscope. However, in other examples, other IMU configurations can be used.

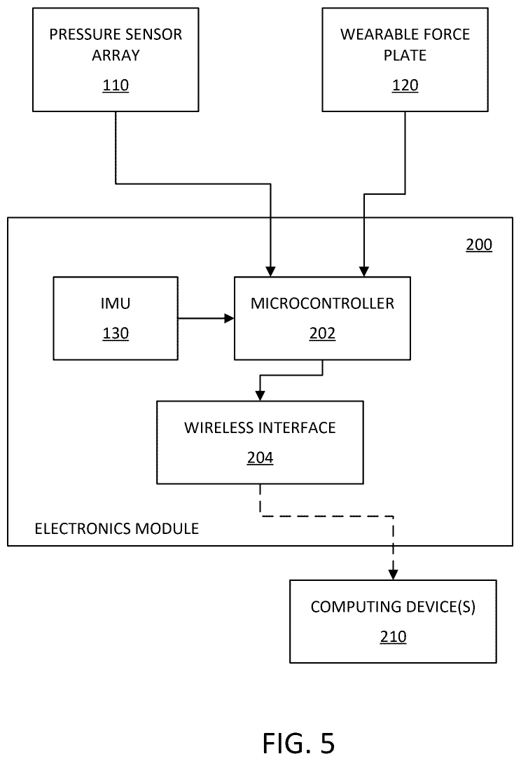

Referring to FIG. 5, in some examples, the IMU 130 is part of an electronics module 200. The electronics module 200 may be integrated into the insole 102, or may be a separate device that can be integrated with or attached to a shoe housing the insole 102, for example. The electronics module 200 may include a microcontroller 202 for collecting sensor data from the flexible pressure sensor array 110, the wearable force plate 120, and/or the IMU 130. The electronics module 200 may further include wireless communication interface 204, such as a BLUETOOTH module, for example, for transmitting sensor data to one or more computing devices 210 for further processing. In some examples, the computing device(s) 210 may execute a smartphone application, or other software application, for processing the sensor data. In some examples, the microcontroller 202 may be include or be part of a calibration module that calibrates the flexible pressure sensor array 110 using sensor data from the flexible pressure sensor array 110, the wearable force plate 120, and/or the IMU 130. In some examples, the one or more computing devices 210 may perform a calibration process for the flexible pressure sensor array 110 using the sensor data from the flexible pressure sensor array 110, the wearable force plate 120, and/or the IMU 130 provided via the wireless interface 204. Accordingly, the one or more computing devices 210 may include or be part of the calibration module.

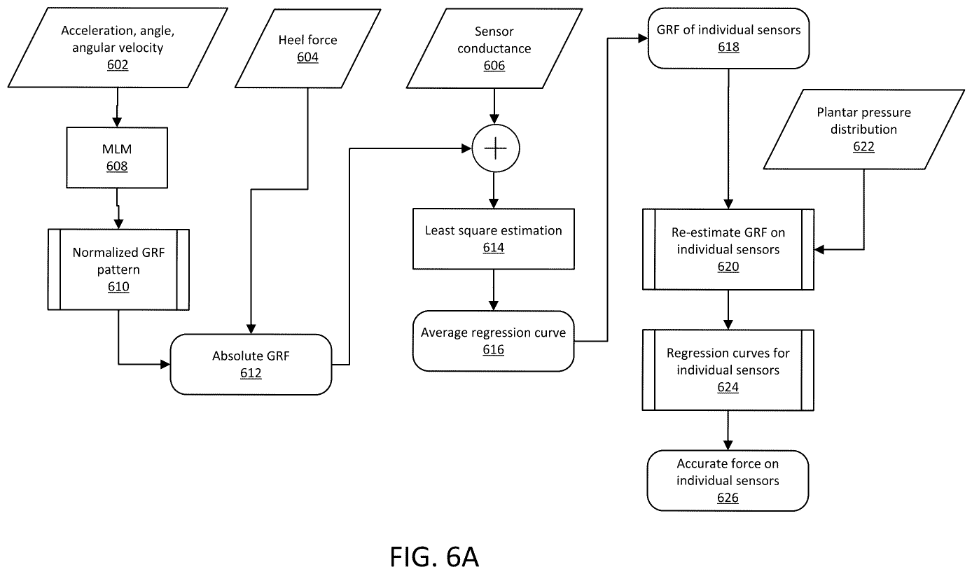

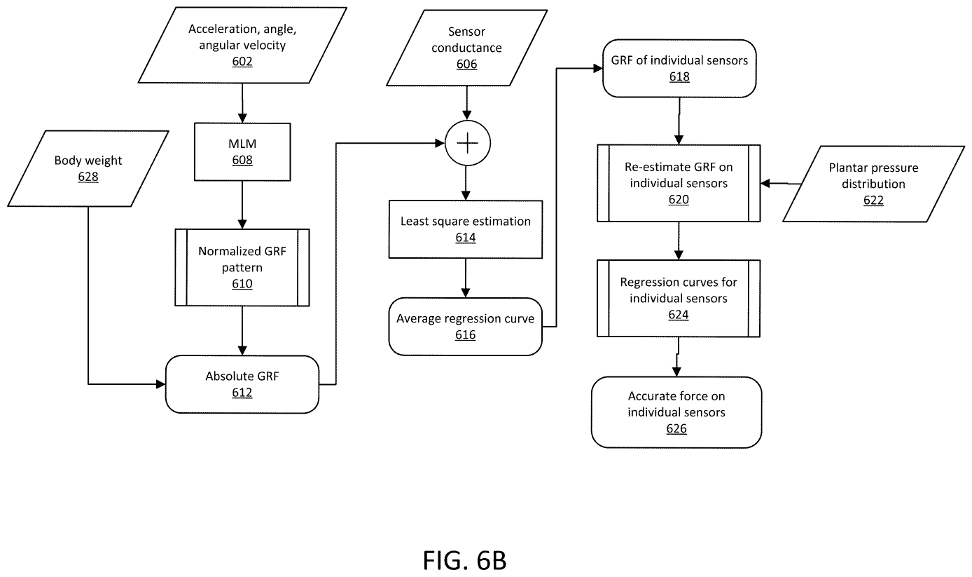

According to certain examples, the wearable force plate 120 introduced into the heel area of the insole 102 Is used to measure the GRF in the heel area. In some examples, a machine-learning model using sensor data from the IMU 130 and optionally the flexible pressure sensor array 110 is configured to estimate the complete GRF during walking. In some examples, the wearable force plate 330 introduced into the heel area of the insole 404 used to measure the GRF in the heel area and to supply reference force measurements that can used as part of the calibration process described below with reference to FIG. 6A. However, in other examples, information such as know body weight can be used instead of the reference force measurements. Accordingly, in some examples, the insole apparatus 100 excludes the wearable force plate 120.

To realize an accurate calibration of each individual sensor 112 of the flexible pressure sensor array 110, a processing technique is applied that includes three steps: estimating an average regression curve for all sensors, re-estimating the true pressure on each sensor during walking, and obtaining the true regression curve of each sensor. This approach eliminates the limitations of manual calibration, making smart insole technologies more accessible for everyday use. Further, it will be appreciated, given the benefit of this disclosure, that although examples below describe performing the calibration during walking, in other examples, the calibration processes can be performed during activities other than walking (such as, but not limited to, running, jumping, standing, etc.).

Examples of an automatic calibration approach are described below with reference to FIGS. 6A and 6B and continuing reference to FIGS. 2-5. Aspects of the calibration method disclosed herein may be implemented on the microcontroller 202 or on a computing device, such as a smartphone or tablet, for example, which may be communicatively coupled to the various sensors via wired/wireless communication links.

According to certain examples, to calibrate the flexible pressure sensor array 110, measurements or estimates of the force over a portion of the foot area or the whole foot area are used. In some examples, foot motion is used to estimate the total pressure based on the close relationship between foot motion and the pressure under foot (as illustrated in FIG. 2, for example). According to certain examples, a calibration process is performed during walking. Walking is a frequently used activity, and the pattern of GRF across the foot during walking motion is relatively simple. These attributes make walking a good activity to use for calibration of the flexible pressure sensor array 110.

Referring to FIG. 6A, a calibration process may use sensor data collected from various components of the insole apparatus 100. For example, the sensor data may include IMU sensor data 602, such as acceleration, angle, and angular velocity, that can be measured by the IMU during walking, for example. During walking, the wearable force plate 120 may measure heel force, and thus, the sensor data used in the calibration process can include sensor data 604 from the wearable force plate 120 (e.g., heel force). For example, the sensor data 604 from the wearable force plate 120 may provide an absolute reference GRF pattern corresponding to the measured heel force during walking. The sensor data may further include sensor data 606 from the flexible pressure sensor array 110, such as sensor conductance measurements, as described further below.

According to certain examples, a machine learning model (MLM), such as an artificial neural network (ANN), can be trained to predict the GRF pattern during walking based on the measurements taken by the IMU (e.g., sensor data 602) and correlations between GRF and foot motion during walking. Accordingly, at operation 608, the machine learning model may produce a normalized GRF pattern 610 that corresponds to whole foot motion during walking. In some examples, the machine learning model can be programmed into one or more processors (e.g., the microprocessor 202) that can be integrated with the insole 102 or positioned external to the insole 102, and communicatively coupled to the various sensors in the insole via one or more wired or wireless communication link(s). In some examples, the machine learning model can be implemented in an application running on a computing device, such as a smartphone, tablet, or other computing device. In such instances, the sensor data 602 may be communicated to the computing device 210 via the wireless interface 204, as described above.

According to certain examples, the machine learning model is implemented as a long short-term memory (LSTM) machine learning model that can be trained to estimate GRF patterns, for example, during walking or other activities. In some examples, the LSTM model can be trained using data acquired from the IMU 130 and center of pressed sensors (COPP) signals from the flexible pressure sensor array 110. Although the COPP signals are obtained from the flexible pressure sensor array 110, the COPP data may remain unaffected by the drifting errors that may be inherent to the flexible pressure sensor array by treating individual pressure sensors 112 in the array as pressed switches to obtain the COPP signal.

Tables 2 through 7 below present experimental performance data for GRF pattern estimation using a trained LSTM. In each example, sensor data was acquired from eight test subjects using an insole having a flexible pressure sensor array and IMU. In Tables 2-7, MSE is mean squared error; RMSE is root mean squared error; R is a measure of the proportion of variance in the dependent variable that can be explained by the independent variable(s) in a regression model; and MAE is mean absolute error in units of body weight. The closer the R\hat{ }2 value is to one, the better the independent variable(s) explain the variability in the dependent variables.

Tables 2-4 present results for an LSTM trained using 80% of the acquired sensor data as training data, 10% of the acquired sensor data as validation data, and 10% of the acquired sensor data as test data (e.g., the data based upon which the trained LSTM produced the results shown in Tables 2-4). Table 2 presents results for the LSTM trained using only IMU sensor data.

TABLE 2
GRF pattern estimation performance for LSTM
trained using IMU data only.

| | Results | | | |
|---|---|---|---|---|
| Test Subject ID | MSE | RMSE | R\hat{ }2 | MAE |
| Subject 1 | 0.0006 | 0.0254 | 0.9969 | 0.0140 |
| Subject 2 | 0.0007 | 0.0263 | 0.9967 | 0.0150 |
| Subject 3 | 0.0007 | 0.0264 | 0.9965 | 0.0147 |
| Subject 4 | 0.0012 | 0.0353 | 0.9933 | 0.0205 |
| Subject 5 | 0.0011 | 0.0332 | 0.9946 | 0.0185 |
| Subject 6 | 0.0009 | 0.0303 | 0.9956 | 0.0170 |
| Subject 7 | 0.0007 | 0.0269 | 0.9964 | 0.0170 |
| Subject 8 | 0.0011 | 0.0334 | 0.9946 | 0.0188 |
| Average | 0.0009 | 0.0296 | 0.9956 | 0.0169 |

Table 3 presents results for the LSTM trained using only COPP data from the flexible pressure sensor array.

TABLE 3
GRF pattern estimation performance for LSTM
trained using COPP data only.

| | Results | | | |
|---|---|---|---|---|
| Test Subject ID | MSE | RMSE | R\hat{ }2 | MAE |
| Subject 1 | 0.0007 | 0.0258 | 0.9968 | 0.0143 |
| Subject 2 | 0.0008 | 0.0276 | 0.9965 | 0.0162 |
| Subject 3 | 0.0004 | 0.0206 | 0.9979 | 0.0118 |
| Subject 4 | 0.0010 | 0.0314 | 0.9946 | 0.0201 |
| Subject 5 | 0.0008 | 0.0286 | 0.9960 | 0.0163 |
| Subject 6 | 0.0006 | 0.0238 | 0.9973 | 0.0140 |
| Subject 7 | 0.0007 | 0.0271 | 0.9964 | 0.0169 |
| Subject 8 | 0.0009 | 0.0302 | 0.9956 | 0.0169 |
| Average | 0.0007 | 0.0269 | 0.9964 | 0.0158 |

Table 4 presents results for the LSTM trained using a combination of IMU sensor data and COPP data.

TABLE 4
GRF pattern estimation performance for LSTM trained
using a combination of IMU data and COPP data.

| | Results | | | |
|---|---|---|---|---|
| Test Subject ID | MSE | RMSE | R\hat{ }2 | MAE |
| Subject 1 | 0.0005 | 0.0219 | 0.9977 | 0.0126 |
| Subject 2 | 0.0006 | 0.0245 | 0.9972 | 0.0142 |
| Subject 3 | 0.0003 | 0.0186 | 0.9982 | 0.0111 |
| Subject 4 | 0.0008 | 0.0278 | 0.9958 | 0.0170 |
| Subject 5 | 0.0007 | 0.0274 | 0.9963 | 0.0155 |
| Subject 6 | 0.0007 | 0.0271 | 0.9964 | 0.0144 |
| Subject 7 | 0.0005 | 0.0232 | 0.9973 | 0.0151 |
| Subject 8 | 0.0007 | 0.0255 | 0.9968 | 0.0147 |
| Average | 0.0006 | 0.0245 | 0.9970 | 0.0143 |

Tables 5-7 present results obtained for each test subject using an LSTM trained based on the acquired sensor data from the other seven test subjects. The sensor data acquired from the instant test subject was used as test data. Thus, for example, the results presented for Subject 1 are based on providing sensor data acquired from Subject 1 to an LSTM that was trained using the sensor data acquired from Subjects 2-8. Table 5 presents results for the LSTM trained using only IMU sensor data.

TABLE 5
GRF pattern estimation performance for LSTM
trained using IMU data only.

| | Results | | | |
|---|---|---|---|---|
| Test Subject ID | MSE | RMSE | R\hat{ }2 | MAE |
| Subject 1 | 0.0029 | 0.0536 | 0.9861 | 0.0315 |
| Subject 2 | 0.0017 | 0.0413 | 0.9921 | 0.0238 |
| Subject 3 | 0.0024 | 0.0486 | 0.9881 | 0.0282 |
| Subject 4 | 0.0029 | 0.0539 | 0.9844 | 0.0325 |
| Subject 5 | 0.0029 | 0.0536 | 0.9859 | 0.0312 |
| Subject 6 | 0.0025 | 0.0497 | 0.9880 | 0.0273 |
| Subject 7 | 0.0022 | 0.0472 | 0.9888 | 0.0279 |
| Subject 8 | 0.0023 | 0.0485 | 0.9886 | 0.0241 |
| Average | 0.0025 | 0.0495 | 0.9878 | 0.0283 |

Table 6 presents results for the LSTM trained using only COPP data from the flexible pressure sensor array.

TABLE 6
GRF pattern estimation performance for LSTM
trained using COPP data only.

| | Results | | | |
|---|---|---|---|---|
| Test Subject ID | MSE | RMSE | R\hat{ }2 | MAE |
| Subject 1 | 0.0023 | 0.0478 | 0.9889 | 0.0262 |
| Subject 2 | 0.0020 | 0.0447 | 0.9907 | 0.0268 |
| Subject 3 | 0.0019 | 0.0431 | 0.9907 | 0.0241 |
| Subject 4 | 0.0038 | 0.0613 | 0.9798 | 0.0374 |
| Subject 5 | 0.0024 | 0.0494 | 0.9880 | 0.0275 |
| Subject 6 | 0.0021 | 0.0456 | 0.9899 | 0.0255 |
| Subject 7 | 0.0017 | 0.0410 | 0.9916 | 0.0254 |
| Subject 8 | 0.0019 | 0.0439 | 0.9906 | 0.0236 |
| Average | 0.0023 | 0.0471 | 0.9888 | 0.0271 |

Table 7 presents results for the LSTM trained using a combination of IMU sensor data and COPP data.

TABLE 7
GRF pattern estimation performance for LSTM trained
using a combination of IMU data and COPP data.

| | Results | | | |
|---|---|---|---|---|
| Test Subject ID | MSE | RMSE | R\hat{ }2 | MAE |
| Subject 1 | 0.00199 | 0.04458 | 0.99040 | 0.02537 |
| Subject 2 | 0.00170 | 0.04125 | 0.99207 | 0.02439 |
| Subject 3 | 0.00165 | 0.04065 | 0.99168 | 0.02300 |
| Subject 4 | 0.00240 | 0.04900 | 0.98707 | 0.03145 |
| Subject 5 | 0.00155 | 0.03941 | 0.99236 | 0.02237 |
| Subject 6 | 0.00136 | 0.03694 | 0.99338 | 0.02142 |
| Subject 7 | 0.00152 | 0.03895 | 0.99242 | 0.02325 |
| Subject 8 | 0.00166 | 0.04077 | 0.99193 | 0.02367 |
| Average | 0.00173 | 0.04145 | 0.99141 | 0.02437 |

Referring again to FIG. 6A, in some examples, a combination of the normalized GRF pattern 610 obtained from the machine learning model (e.g., using a trained LSTM as described above) at operation 608 and reference heel force measurements (sensor data 604) obtained from the wearable force plate 120 can be used to estimate an absolute GRF 612 during walking. For example, the absolute GRF 612 may be estimated by using the reference heel force measurements from the wearable force plate 120 to convert the normalized GRF pattern 610 produced by the machine learning model at operation 608 into units of force (e.g., Newtons).

Using the estimated GRF pattern from the machine learning model (trained using any combination of sensor data as described above) in combination with reference force measurements from the wearable force plate 120 to produce the absolute estimated GRF 612 may provide a high accuracy method by which to calibrate the flexible pressure sensor array 110 according to processes described herein. However, in other examples, automatic calibration of the flexible pressure sensor array 110 may be accomplished without using the wearable force plate 120. For example, referring to FIG. 6B, in some examples, the absolute estimated GRF 612 can be obtained using body weight information 628 (e.g., a known body weight of an individual wearing the insole apparatus 100) to convert the normalized GRF pattern 610 into units of force. In some examples, omitting the wearable force plate 120 may increase cost-efficiency and comfort associated with the insole apparatus 100.

Thus, in some examples, the insole apparatus 100 includes the flexible pressure sensor array 110, the IMU 130, and the wearable force plate 120. An automatic calibration process for the flexible pressure sensor array 110 may include training an LSTM machine learning model with COPP and IMU sensor data, and using the trained LSTM machine learning model to estimate the normalized GRF pattern 610. The calibration process may further include using reference force measurements from the wearable force plate 120 to estimate the GRF in Newtons (e.g., produce the absolute estimated GRF 612 as described above). This approach may provide a very high accuracy calibration, in combination with the remaining process steps described below.

In other examples, the insole apparatus 100 includes the flexible pressure sensor array 110, the IMU 130 (omitting the wearable force plate 120). In some such examples, an automatic calibration process for the flexible pressure sensor array 110 may include training an LSTM machine learning model with COPP and IMU sensor data, and using the trained LSTM machine learning model to estimate the normalized GRF pattern 610. The calibration process may further include using known body weight information to estimate the GRF in Newtons (e.g., produce the absolute estimated GRF 612 as described above). This approach (in combination with process steps described below) may achieve calibration of the flexible pressure sensor array 110 with good accuracy.

In further examples, the insole apparatus 100 includes the flexible pressure sensor array 110 (omitting the wearable force plate 120 and the IMU 130). In some such examples, an automatic calibration process for the flexible pressure sensor array 110 may include training an LSTM machine learning model with COPP data, and using the trained LSTM machine learning model to estimate the normalized GRF pattern 610. The calibration process may further include using known body weight information to estimate the GRF in Newtons (e.g., produce the absolute estimated GRF 612 as described above). This approach (in combination with process steps described below) still may achieve calibration of the flexible pressure sensor array 110 with good accuracy.

In some examples, the estimated absolute GRF 612 may be an estimate of the GRF for the whole foot during walking. However, to calibrate the flexible pressure sensor array 110, estimates of the GRF for regions of the insole 102 corresponding to different regions of the foot (e.g., heel, mid-foot, forefoot), estimates of the GRF measured by individual sensors in the array, and/or estimates of the GRF during activities other than walking, may be needed. Accordingly, in certain examples, sensor measurements are obtained from the flexible pressure sensor array 110 to produce the sensor data 606 that may be used to produce estimated GRF for regions of the foot area and/or individual sensors 112. In one example, the sensor measurements are sensor conductance measurements; however, in other configurations, sensor resistance or other measurements may be used.

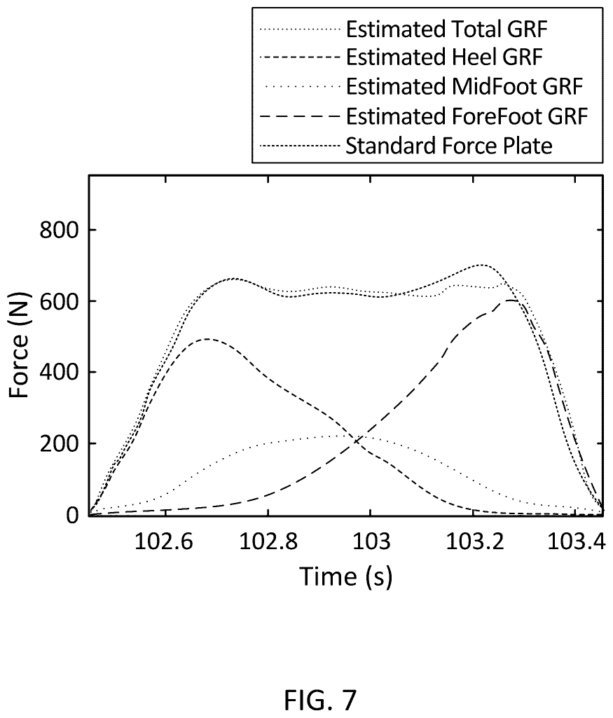

Referring to FIGS. 6A and 6B, based on an assumption that only sensors 112 in the same foot area (e.g., heel, mid-foot, forefoot) have the same average regression curve, in one example, three different regression curves can be estimated for sensors 112 in the heel, mid-foot, and forefoot areas, respectively. At operation 614, a least squares estimation may be applied to a combination of the sensor data 606 from the flexible pressure sensor array 110 (e.g., sensor conductance measurements) and at least a corresponding portion of the estimated absolute GRF 612 to produce, at operation 616, an average regression curve for different foot regions (e.g., heel, mid-foot, forefoot). For example, sensor measurements from the heel region of the insole 404/flexible pressure sensor array 310 in combination with the estimated absolute GRF 612 for the heel region of the can be used to produce an average regression curve of the heel sensors. Similarly, sensor measurements from the individual sensors 312 in other foot regions (e.g., forefoot/toe, mid-foot, etc.) and corresponding portions of the estimated absolute GRF 612 can be used to produce average regression curves for those groups of sensors. In other examples, operation 614 may include using an estimation technique other than least squares estimation. Based on the average regression curves of different foot regions, estimated GRF corresponding to the different foot regions can be produced during all (or at least several) activities, rather than only walking. The summation of all the regional GRF is the complete GRF under foot, as shown in FIG. 7, for example. The estimated regional GRF curves are produced based on the average regression curves of different regions, determined at operation 616, and their summation is the complete total GRF under foot.

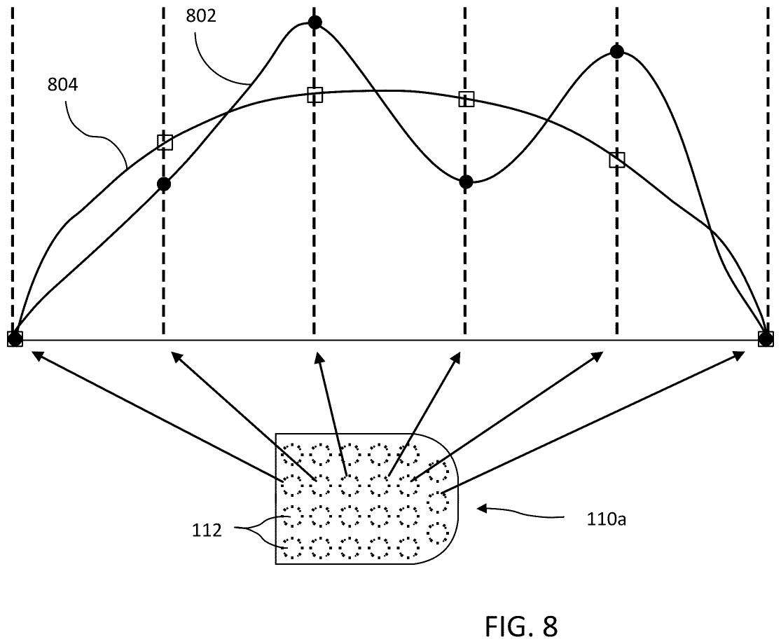

At operation 618, an estimate of the GRF at some or all individual sensors 112 in the flexible pressure sensor array 110 can be obtained. This information can then be used to calibrate the flexible pressure sensor array 110. According to certain examples, information about the geometry of the foot can be used to adjust the average regression curve obtained at operation 616 for individual sensors 112 within the flexible pressure sensor array 110. For example, known information about the plantar pressure in the heel area, and/or other areas of the foot (e.g., plantar pressure distribution information 622), can be used to adjust the regression curve values for individual sensors 112. For example, FIG. 8 illustrates a curve 802 representing estimated sensor pressure for a heel region of the foot based on an average regression curve produced at operation 616. Referring to FIGS. 6 and 8, at operation 620, GRF values for individual sensors 112 in the corresponding portion 110a of the insole 102 can be adjusted to produce a more accurate estimate of the GRF at the individual sensors. FIG. 8 further illustrates a curve 804 showing re-estimated sensor pressure (force) measurements for sensors 112 in the portion 110a of the insole 102. These force estimates can then be used to calibrate the flexible pressure sensor array 110. For example, as shown in FIGS. 6A and 6B, at operation 624, regression curves for individual sensors 112 can be produced based on the re-estimated GRF values at the individual sensors (obtained at operation 622). Using the individual regression curves obtained at operation 624, an accurate estimate of the force at each individual sensor can be obtained at operation 626.

Thus, aspects and embodiments provide a “smart” insole and a calibration method that can be used to automatically calibrate a pressure sensor array within the insole during a common activity, such as walking, without requiring special equipment and without requiring the person wearing the insole to perform any special activity or process. The calibration procedure can be automatically repeated and may therefore ensure long-term, measurement accuracy of the flexible pressure sensor array. With accurate force measurements obtained from the calibrated flexible pressure sensor array, the insole can be used for a wide variety of applications, including pressure monitoring for early detection of conditions such as diabetic foot ulcers, or other foot-related health concerns, as well as lifted load weight monitoring which may assist in preventing back injuries.

The following examples pertain to further embodiments, from which numerous permutations and configurations will be apparent.

Example 1 is an insole apparatus comprising: an insole; a flexible pressure sensor array coupled to the insole and including a plurality of pressure sensors distributed over a surface area of the insole; a force plate integrated into a heel region of the insole; and an inertial measurement unit coupled to the insole.

Example 2 includes the insole apparatus of Example 1, wherein the force plate includes a plurality of micro load cells.

Example 3 includes the insole apparatus of Example 2, wherein the plurality of micro load cells includes four micro load cells, one positioned at each corner of the force plate.

Example 4 includes the insole apparatus of any one of Examples 1-3, wherein the force plate is configured to provide reference force measurements.

Example 5 includes the insole apparatus of Example 4, further comprising a calibration module communicatively coupled to the flexible pressure sensor array, the force plate, and the inertial measurement unit.

Example 6 includes the insole apparatus of Example 5, wherein, during walking motion of the insole, the calibration module is configured to: estimate a ground reaction force (GRF) distribution for the heel region of the insole based on the reference force measurements from the force plate; produce a normalized GRF walking pattern based on measurements from the inertial measurement unit; determine an absolute GRF walking pattern based on the GRF distribution for the heel region of the insole, the normalized GRF walking pattern, and/or force measurements from the flexible pressure sensor array; and determine an average regression curve based on the absolute GRF walking pattern using least squares estimation.

Example 7 includes the insole apparatus of Example 6, wherein the calibration module is further configured to: estimate a GRF distribution between individual sensors of the flexible pressure sensor array based on foot geometry; re-estimate the force of the individual sensors calculated by the average regression curves based on the GRF distribution between the individual sensors; and calibrate the force measurements for the individual sensors by producing individual regression curves for the respective individual sensors, based on the re-estimated force of individual sensors.

Example 8 includes the insole apparatus of one of Examples 6 or 7, wherein the calibration module includes one or more processors configured to operate an artificial neural network to produce the normalized GRF walking pattern.

Example 9 includes the insole apparatus of any one of Examples 5-8, wherein the calibration module includes a microcontroller disposed on the insole.

Example 10 includes the insole apparatus of any one of Examples 5-8, wherein the calibration module is implemented in a computing device communicatively coupled to the flexible pressure sensor array, the force plate, and the inertial measurement unit via a wireless communications interface.

Example 10 is a method of calibrating a pressure sensor array in an insole during a walking activity, the method comprising: estimating a ground reaction force (GRF) distribution for the heel region of the insole based on reference force measurements from a force plate embedded in the heel region of the insole; producing a normalized GRF walking pattern based on measurements from an inertial measurement unit coupled to the insole; determining an absolute GRF walking pattern based on the GRF distribution for the heel region of the insole, the normalized GRF walking pattern, and/or force measurements from the pressure sensor array; determining average regression curves based on the absolute GRF during walking using least squares estimation; estimating a GRF distribution between individual sensors of the pressure sensor array based on foot geometry; re-estimating the force of the individual sensors calculated by the average regression curves based on the GRF distribution between the individual sensors; and calibrating the force measurements for the individual sensors by producing individual regression curves for the respective individual sensors based on the re-estimated force of the individual sensors.

Example 11 is an insole apparatus including at least one processor configured to implement the method of Example 10.

Example 12 is an insole apparatus comprising: an insole; a flexible pressure sensor array coupled to the insole and including a plurality of pressure sensors distributed over a surface area of the insole, the flexible pressure sensor array configured to produce pressure sensor data; a force plate integrated into a heel region of the insole and configured to reference force measurements; an inertial measurement unit (IMU) configured to produce IMU sensor data; and at least one processor configured to implement a calibration procedure for the flexible pressure sensor array based on the pressure sensor data, the IMU sensor data, and the reference force measurements.

Example 13 includes the insole apparatus of Example 12, wherein to implement the calibration procedure, the at least one processor is configured to operate a trained machine learning model to produce a normalized ground reaction force (GRF) pattern based on at least one of the IMU sensor data or the pressure sensor data.

Example 14 is an insole apparatus comprising: an insole; a flexible pressure sensor array coupled to the insole and including a plurality of pressure sensors distributed over a surface area of the insole, the flexible pressure sensor array configured to produce pressure sensor data, the pressure sensor data including force measurements from individual pressure sensors in the flexible pressure sensor array; and at least one processor configured to implement the calibration process for the flexible pressure sensor array. The calibration process comprises: applying a machine learning model to produce an estimated normalized ground reaction force (GRF) pattern for a whole foot region corresponding to the insole, converting the estimated normalized GRF pattern to units of force to produce an estimated absolute whole foot GRF pattern, determining an average regression curve based on the absolute whole foot GRF pattern using least squares estimation, based on the average regression curve, estimating GRF values for the individual pressure sensors of the flexible pressure sensor array, adjusting the estimated GRF values based on based on foot geometry information to produce regression curves for the individual pressure sensors, and calibrating the force measurements from the individual sensors based on the regression curves.

Example 15 includes the insole apparatus of Example 14, wherein applying the machine learning model to produce an estimated normalized GRF pattern includes applying a machine learning model that has been trained using the pressure sensor data.

Example 16 includes the insole apparatus of one of Examples 14 or 15, further comprising an inertial measurement unit (IMU) configured to produce IMU sensor data.

Example 17 includes the insole apparatus of Example 16, wherein applying the machine learning model to produce an estimated normalized GRF pattern includes processing the IMU sensor data with the machine learning model to produce the estimated normalized GRF pattern based on the IMU sensor data.

Example 18 includes the insole apparatus of Example 17, wherein applying the machine learning model to produce the estimated normalized GRF pattern includes applying a machine learning model that has been trained using the IMU sensor data.

Example 19 includes the insole apparatus of any one of Examples 14-18, wherein converting the estimated normalized GRF pattern to units of force includes using body weight of a wearer of the insole to convert the estimated normalized GRF pattern to units of force.

Example 20 includes the insole apparatus of any one of Examples 14-18, further comprising a force plate integrated into a heel region of the insole and configured to provide reference force measurements.

Example 21 includes the insole apparatus of Example 20, wherein converting the estimated normalized GRF pattern to units of force includes converting the estimated normalized GRF pattern to units of force based on the reference force measurements.

Example 21 includes the insole apparatus of one of Examples 20 or 21, wherein the force plate includes a plurality of micro load cells disposed within a housing.

Example 22 includes the insole apparatus of any one of Examples 14-21, wherein the at least one processor includes a microcontroller coupled to the insole.

Example 23 includes the insole apparatus of any one of Examples 14-21, further comprising: a microcontroller communicatively coupled to the flexible pressure sensor array and optionally to the inertial measurement unit and/or to the force plate; and a wireless communication interface coupled to the microcontroller; wherein the microcontroller is configured to transmit the pressure sensor data, and optionally the IMU sensor data and/or the reference force measurements, to an external computing device via the wireless communication interface; and wherein the external computing device comprises the at least one processor.

Example 24 is a method of calibrating a flexible pressure sensor array in an insole, the method comprising applying a machine learning model to produce an estimated normalized ground reaction force (GRF) pattern for a whole foot region corresponding to the insole; converting the estimated normalized GRF pattern to units of force to produce an estimated absolute whole foot GRF pattern; determining an average regression curve based on the absolute whole foot GRF pattern using least squares estimation; based on the average regression curve, estimating GRF values for the individual pressure sensors of the flexible pressure sensor array; adjusting the estimated GRF values based on based on foot geometry information to produce regression curves for the individual pressure sensors; and calibrating force measurements from the individual sensors based on the regression curves.

Having described above several aspects of at least one embodiment, it is to be appreciated various alterations, modifications, and improvements will readily occur to those skilled in the art. Such alterations, modifications, and improvements are intended to be part of this disclosure and are intended to be within the scope of the invention. Accordingly, the foregoing description and drawings of various embodiments are presented by way of example only. These examples are not intended to be exhaustive or to limit the invention to the precise forms disclosed. The methods and apparatuses are capable of implementation in other embodiments and of being practiced or of being carried out in various ways. In addition, the phraseology and terminology used herein is for the purpose of description and should not be regarded as limiting. Any references to examples, components, elements, or acts of the systems and methods herein referred to in the singular can also embrace examples including a plurality, and any references in plural to any example, component, element or act herein can also embrace examples including only a singularity. References in the singular or plural form are not intended to limit the presently disclosed systems or methods, their components, acts, or elements. The use herein of “including”, “comprising”, “having”, “containing”, “involving”, and variations thereof is meant to encompass the items listed thereafter and equivalents thereof as well as additional items. References to “or” can be construed as inclusive so that any terms described using “or” can indicate any of a single, more than one, and all of the described terms.

1. An insole apparatus comprising:
an insole;
a flexible pressure sensor array coupled to the insole and including a plurality of pressure sensors distributed over a surface area of the insole;
a force plate integrated into a heel region of the insole and configured to provide reference force measurements;
an inertial measurement unit coupled to the insole; and
a calibration module communicatively coupled to the flexible pressure sensor array, the force plate, and the inertial measurement unit, wherein during walking motion of the insole, the calibration module is configured to:
estimate a ground reaction force (GRF) distribution for the heel region of the insole based on the reference force measurements from the force plate,
produce a normalized GRF walking pattern based on measurements from the inertial measurement unit,
determine an absolute GRF walking pattern based on a combination of the GRE distribution for the heel region of the insole and the normalized GRF walking pattern, and
determine an average regression curve based on the absolute GRF walking pattern using least squares estimation.

2. The insole apparatus of claim 1, wherein the force plate includes a plurality of micro load cells.

3. (canceled)

4. (canceled)

5. (canceled)

6. The insole apparatus of claim 1, wherein the calibration module is further configured to:
estimate a GRF distribution between individual sensors of the flexible pressure sensor array based on foot geometry;
re-estimate the force of the individual sensors calculated by the average regression curves based on the GRF distribution between the individual sensors; and
calibrate the force measurements for the individual sensors by producing individual regression curves for the respective individual sensors, based on the re-estimated force of individual sensors.

7. The insole apparatus of claim 1, wherein the calibration module includes one or more processors configured to operate an artificial neural network to produce the normalized GRF walking pattern.

8. The insole apparatus of claim 1, wherein the calibration module includes a microcontroller disposed on the insole.

9. The insole apparatus of claim 1, wherein the calibration module is implemented in a computing device communicatively coupled to the flexible pressure sensor array, the force plate, and the inertial measurement unit via a wireless communications interface.

10. An insole apparatus comprising:
an insole;
a flexible pressure sensor array coupled to the insole and including a plurality of pressure sensors distributed over a surface area of the insole, the flexible pressure sensor array configured to produce pressure sensor data, the pressure sensor data including force measurements from individual pressure sensors in the flexible pressure sensor array; and
at least one processor configured to implement the calibration process for the flexible pressure sensor array, wherein the calibration process comprises
applying a machine learning model to produce an estimated normalized ground reaction force (GRF) pattern for a whole foot region corresponding to the insole,
converting the estimated normalized GRF pattern to units of force to produce an estimated absolute whole foot GRF pattern,
determining an average regression curve based on the absolute whole foot GRF pattern using least squares estimation,
based on the average regression curve, estimating GRF values for the individual pressure sensors of the flexible pressure sensor array,
adjusting the estimated GRF values based on based on foot geometry information to produce regression curves for the individual pressure sensors, and
calibrating the force measurements from the individual sensors based on the regression curves.

11. The insole apparatus of claim 10, wherein applying the machine learning model to produce an estimated normalized GRF pattern includes applying a machine learning model that has been trained using the pressure sensor data.

12. The insole apparatus of claim 10, further comprising
an inertial measurement unit (IMU) configured to produce IMU sensor data.

13. The insole apparatus of claim 12, wherein applying the machine learning model to produce an estimated normalized GRF pattern includes processing the IMU sensor data with the machine learning model to produce the estimated normalized GRF pattern based on the IMU sensor data.

14. The insole apparatus of claim 13, wherein applying the machine learning model to produce the estimated normalized GRF pattern includes applying a machine learning model that has been trained using the IMU sensor data.

15. The insole apparatus of claim 10, wherein converting the estimated normalized GRF pattern to units of force includes using body weight of a wearer of the insole to convert the estimated normalized GRF pattern to units of force.

16. The insole apparatus of claim 10, further comprising:
a force plate integrated into a heel region of the insole and configured to provide reference force measurements.

17. The insole apparatus of claim 16, wherein converting the estimated normalized GRF pattern to units of force includes converting the estimated normalized GRF pattern to units of force based on the reference force measurements.

18. The insole apparatus of claim 10, wherein the at least one processor includes a microcontroller coupled to the insole.

19. The insole apparatus of claim 10, further comprising:
a microcontroller communicatively coupled to the flexible pressure sensor array; and
wireless communication interface coupled to the microcontroller;
wherein the microcontroller is configured to transmit the pressure sensor data to an external computing device via the wireless communication interface; and
wherein the external computing device comprises the at least one processor.

20. A method of calibrating a pressure sensor array in an insole during a walking activity, the method comprising:
estimating a ground reaction force (GRF) distribution for the heel region of the insole based on reference force measurements from a force plate embedded in the heel region of the insole;
producing a normalized GRF walking pattern based on measurements from an inertial measurement unit coupled to the insole;
determining an absolute GRF walking pattern based on the GRF distribution for the heel region of the insole, the normalized GRF walking pattern, and/or force measurements from the pressure sensor array;
determining average regression curves based on the absolute GRF during walking using least squares estimation;
estimating a GRF distribution between individual sensors of the pressure sensor array based on foot geometry;
re-estimating the force of the individual sensors calculated by the average regression curves based on the GRF distribution between the individual sensors; and
calibrating the force measurements for the individual sensors by producing individual regression curves for the respective individual sensors based on the re-estimated force of the individual sensors.

21. The method of claim 20, wherein determining the absolute GRF walking pattern comprises determining the absolute GRF walking pattern based on a combination of the GRF distribution for the heel region of the insole and the normalized GRF walking pattern.

22. The method of claim 20, wherein producing the normalized GRF walking pattern comprises operating an artificial neural network to produce the normalized GRF walking pattern based on the measurements from the inertial measurement unit.
